(19) United States
(12) Patent Application Publication
(10) Pub. No.: US 2009/0182348 A1
(43) Pub. Date: Jul. 16, 2009

(54) EMITTER POSITIONING AND HOLDING AUTOMATED DEVICE FOR MEDICAL SYSTEMS USING EMITTER/SENSOR SYSTEM

(76) Inventors: Gevorg Nahapetian, Pleasanton, CA (US); Kambiz Behzadi, Pleasanton, CA (US)

Correspondence Address: GEVORG NAHAPETIAN, 1423 MAPLE LEAF COURT, PLEASANTON, CA, 94588, US

(21) Appl. No.: 12/013,353

(22) Filed: Jan. 11, 2008

(51) Int. Cl. A61B 19/00

(52) U.S. Cl. 606/130

(57) ABSTRACT

The present invention includes controller coupled with actuators, swivel balanced arm holder with rotating head, location position information system that can maintain emitter of medical Emitter/Sensor Positioning System in desired position. This enables surgeon to reduce number of personnel in operating room, reduces the time of the surgery, makes incisions and cuts more precise.

### BACKGROUND

During surgeries surgeons are using emitter/sensor positioning system to properly locate/identify relative position of various body parts and surgical equipment, this enables the surgeon to perform correct incisions/cuts which increases the chance of successful operation and reduces the after surgery recovery time. Typical emitter /sensor positioning system is shown in FIG. 1.

In this system 100 Sensors 106 and 108 are placed on the body parts or medical equipment. Emitter 102 emits signal 104 (electrical, electromagnetic, optical, radioactive, etc.),

Multiple Sensors 106 108 sense the strength and type of signal and feed that signal information to Location/Position/Information System 110 (LPIS). LPIS 110 calculates/displays information about relative location of the Sensors 106 108. Based on that information surgeon can perform medical device orientation, incision and cut location and direction.

In this system position of the emitter 102 is critical, because detected signal strength by sensors 106 and 108 depends on it. Emitter 102 must be easily moved because of the movement and flexibility required during the operation and varying size and location of the patient. However it must maintain steady position to maintain accurate signal 104 for sensor positions.

Presently to maintain proper position of the emitter 102, surgeon's assistant holds it and moves it as required to maintain proper signal for sensors 106 108. He has to follow movement of body parts or medical devices to which sensors 106 108 are attached. When there is no movement, he has to hold emitter 102 steady/motionless to prevent faulty location/position information. Alternatively there is an articulate lockable arm that holds emitter 102 in one position only, then surgeon has to unlock it to move to new position to follow the movement of the body parts or medical device, then he has to lock it again. Advantage of the lockable arm is that it is steady for motionless mode, but because it's lockable feature it is very hard to use when there are multiple motions of patient or medical device. Our invention reduces the surgery time, and recovery time for the patient.

### BRIEF DESCRIPTION OF THE DRAWINGS

FIG. 1 is a block diagram of typical emitter/sensor positioning system (existing setup)

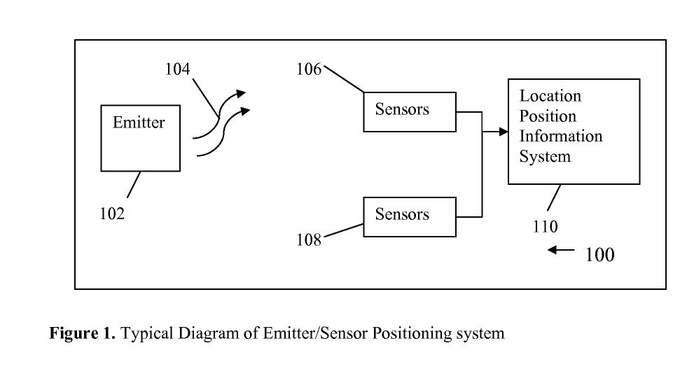

FIG. 2 is a block diagram of our invention with Emitter Positioning and Holding Automated Device System 200.

FIG. 3 is a block diagram of our invention with Control System for Automated Emitter Positioning and Holding Device System 300.

### DETAILED DESCRIPTION

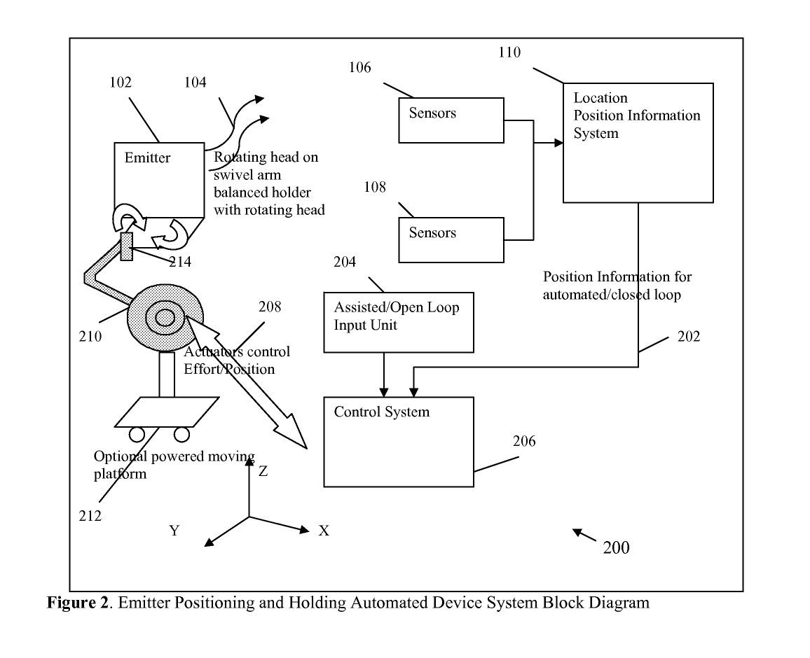

We invented Emitter Positioning and Holding Automated Device System 200. It consists of Swivel Balanced Arm Holder 210 with Rotating Head 214, Actuators 208, and Control System 206 (CS). Control System 206 reads Signals 202 from LPIS 110 and from Actuators 208 and/or from Assisted Input Unit 204 and controls Actuators 208 to set Emitter 102 into desired position. Diagram of the system is shown in FIG. 2.

Swivel Balanced Arm 210 provides ability to adjust position of the head which holds Emitter 102 in X/Y/Z directions, since it is balanced it also can be used without actuators or control system in free motion/manual mode.

Swivel Arm 210 can be mounted stationary near surgery table, or be on Powered Moving (wheeled) Platform 212 to provide even greater flexibility to adjust position of the holder in X/Y directions. Rotating Head 214 provides ability to adjust the angles along the X/Y/Z axis to maintain proper angles when Swivel Arm 210 moves.

Assisted Open Loop Input 204 gives ability to move Emitter 102 into desired position by manually entering the desired location, it has individual controls for each coordinate X/Y/Z and each angle for the Balanced Arm Holder 210 and Rotating Head 214.

Control System 206 runs multiple proportional/integral/differential PID controllers to control actuators to achieve desired position of the Emitter 102, based on the input either from LPIS 110 or Assisted/Open Loop input 204.

Actuators 208 can move Swivel Arm 210 and Rotating Head 214 into desired position. They can be simple hydraulic/pneumatic systems with valves, or electric DC/AC/permanent magnet motors or any other device that can move the portions of Swivel Arm 210 and Rotating Head 214.

### Modes of Operation

Our system will operate in three distinct modes Manual, Semi-Automated, Automated.
1. In Manual mode all Actuators 208 and Control System 206 are disabled. Emitter holder can be moved by simply moving the Holder Rotating Head 214, since Swivel Arm 210 is balanced, Emitter 102 will be stable at any position it is moved.
2. In Semi-Automated mode Control System 206 controls Actuators 208 to the position based on the Assisted/Open Loop Input (AOLI) 204. Surgeon has individual control of each degree of freedom for the holder and can request the direction and amount of the motion. Control System 206 will translate the request from AOLI 204 into various effort for each Actuator 208 and activate them to achieve requested motion like up/down, left/right, tilt/rotate.
3. In Automated mode Control System 206 moves Actuators 208 constantly to achieve optimized signals at Sensors 106 108.

### Control System and Algorithms

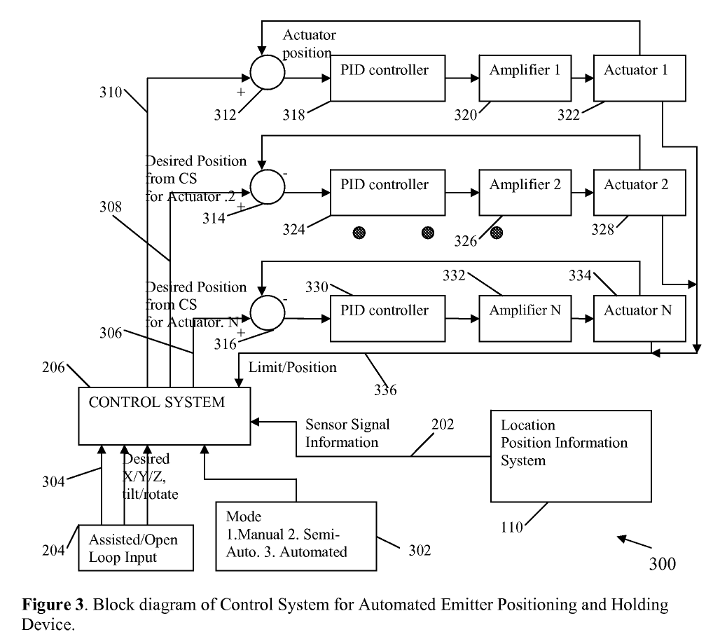

Diagram describing the Control System operation 300 is shown in FIG. 3. Control System 206 uses microprocessor to perform activation of the each Actuator 322 328 334 based on the data from LPIS 110 and AOLI 204 and Actuators 322 328 334 actual position. Since the Swivel Arm 210 has various Actuators and their motion is not directly translated into X/Y/Z coordinates, Control System 206 calculates/translates desired position into control effort for each Actuator. It also employs the final element analysis and Limit Sensors 336 to prevent collision between Emitter 102 and surrounding objects (patient, equipment, personnel, etc.). It also reads back relative position 336 of each Actuator so coordinate translation can be done.

Each Actuator 322 328 334 has its own control loop to control its effort/position. Control System 206 gets input in which mode to operate from Mode Input 302. It calculates Desired Position 310 308 306 for each Actuator 322 328 334. That desired position is fed into Proportional Integral Differential (PID) controllers 318 324 330 via at Error Calculating Blocks 312 314 316, which subtracts actual position for each actuator from the desired position. PID controllers 318 324 330 calculate effort required to move the Actuators 322 328 334. That effort gets amplified by Amplifiers 322 326 332 which will perform the move of Actuators 322 328 334. Amplifiers 320 326 332 can be various type like hydraulic, pneumatic, electric motors, etc.

What we claim:

1. An apparatus comprising of emitter, emitter holder, controller, actuators, sensors, location position information system, which can place emitter into desired position using swivel balanced arm with rotating head in manual or automatic mode.

2. Actuators enable hands free movement of the holder with Assisted/Open loop modes of operation.

3. Microprocessor controlled control system enables quick translations of desired X/Y/Z/tilt/rotate requests into proper efforts for each actuator and enables tracking motion of the body parts and medical equipment to maintain optimized signals from sensors
